(19) United States
(12) Patent Application Publication
(10) Pub. No.: US 2023/0131539 A1
(43) Pub. Date: Apr. 27, 2023

(54) SYSTEMS AND METHODS FOR A PET RELATIVE FINDER

(71) Applicant: Mars, Incorporated, McLean, VA (US)

(72) Inventors: Daniel GARRIGAN, Portland, OR (US); Jason Troy HUFF, Portland, OR (US); Rebecca Chodroff FORAN, Camas, WA (US)

(21) Appl. No.: 18/049,799

(22) Filed: Oct. 26, 2022

Related U.S. Application Data
(60) Provisional application No. 63/263,138, filed on Oct. 27, 2021.

(51) Int. Cl. G16B 40/00; G16B 30/00

(52) U.S. Cl. CPC G16B 40/00; G16B 30/00

(57) ABSTRACT

A computer-implemented method for using companion pet DNA information to train a machine-learning model to predict a familial relationship of a companion pet, the method comprising receiving companion pet DNA information, the companion pet DNA information including at least one DNA sequence, at least one matching companion pet, and at least one familial label corresponding to the familial relationship between the companion pet and the at least one matching companion pet, and upon the receiving, training the machine-learning model to predict at least one familial relationship from the at least one DNA sequence.

### CROSS-REFERENCE TO RELATED APPLICATION(S)

This patent application claims the benefit of priority to U.S. Provisional Application No. 63/263,138, filed Oct. 27, 2021, which is incorporated herein by reference in its entirety.

### TECHNICAL FIELD

Various embodiments of this disclosure relate generally to machine-learning based techniques for predicting a familial relationship of a companion pet. In some embodiments, the disclosure relates to systems and methods for training a machine-learning based model to predict a familial relationship of a companion pet.

### BACKGROUND

Determining a companion pet's familial relationships is an incredibly challenging and error-prone process. For example, conventional techniques may include comparing similar DNA fragments of two companion pets to determine if such companion pets are related. If the two companion pets share similar DNA, conventional techniques may conclude that the two companion pets are related. However, conventional techniques fail to distinguish between DNA fragments that indicate inbreeding and DNA fragments that indicate a familial relationship. As a result, conventional techniques determine that two companion pets are related, when the companion pets have actually been inbred. Furthermore, conventional techniques lack the capability to provide a user with a familial relationship label (e.g., mother, sister, half-sister, nephew, etc.) of two or more companion pets.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for predicting a familial relationship of a companion pet.

In one aspect, an exemplary embodiment of a method for using companion pet DNA information for training a machine-learning system to predict a familial relationship of a companion pet may include receiving companion pet DNA information, the companion pet DNA information including at least one DNA sequence, at least one matching companion pet, and at least one familial label corresponding to the familial relationship between the companion pet and the at least one matching companion pet, and upon the receiving, training the machine-learning model to predict at least one familial relationship from the at least one DNA sequence. The training may further include, for each of the at least one DNA sequence, extracting at least one local DNA fragment pattern from a database, the at least one local DNA fragment pattern corresponding to the at least one matching companion pet, analyzing the at least one local DNA fragment pattern to determine at least one predicted familial relationship between the companion pet and the at least one matching companion pet, and based on the analyzing, determining at least one predicted familial relationship and at least one corresponding confidence level.

In a further aspect, an exemplary embodiment of a computer system for using companion pet DNA information to train a machine-learning model to predict a familial relationship of a companion pet may include at least one memory storing instructions and at least one processor configured to execute the instructions to perform operations. The operations may include receiving companion pet DNA information, the companion pet DNA information including at least one DNA sequence, at least one matching companion pet, and at least one familial label corresponding to a relationship between the companion pet and the at least one matching companion pet, and upon the receiving, training the machine-learning model to predict at least one familial relationship from the at least one DNA sequence. The training may further include, for each of the at least one DNA sequence, extracting at least one local DNA fragment pattern from a database, the at least one local DNA fragment pattern corresponding to the at least one matching companion pet, analyzing the at least one local DNA fragment pattern to determine at least one predicted familial relationship between the companion pet and the at least one matching companion pet, and based on the analyzing, determining at least one predicted familial relationship and at least one corresponding confidence level.

In a further aspect, a non-transitory computer-readable medium may contain instructions that, when executed by a processor, cause the processor to perform operations for using companion pet DNA information to train a machine-learning model to predict a familial relationship of a companion pet. The operations may include receiving companion pet DNA information, the companion pet DNA information including at least one DNA sequence, at least one matching companion pet, and at least one familial label corresponding to a relationship between the companion pet and the at least one matching companion pet, and upon the receiving, training the machine-learning model to predict at least one familial relationship from the at least one DNA sequence. The training may further include, for each of the at least one DNA sequence, extracting at least one local DNA fragment pattern from a database, the at least one local DNA fragment pattern corresponding to the at least one matching companion pet, analyzing the at least one local DNA fragment pattern to determine at least one predicted familial relationship between the companion pet and the at least one matching companion pet, and based on the analyzing, determining at least one predicted familial relationship and at least one corresponding confidence level.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIGS. 1A-1J describe exemplary environments of a platform for predicting a familial relationship of a companion pet, according to one or more embodiments.

FIG. 2 depicts a flowchart of an exemplary method of training a machine-learning model to predict a familial relationship of a companion pet, according to one or more embodiments.

FIG. 3 depicts a flowchart of an exemplary method for training a machine-learning model to analyze the at least one local DNA fragment pattern to determine at least one predicted familial relationship, according to one or more embodiments.

FIG. 4 depicts a flowchart further illustrating training the machine-learning model to predict at least one shared trait between a companion pet and a matching companion pet, according to one or more embodiments.

FIG. 5 depicts a flowchart further illustrating training the machine-learning model to output a list of matching DNA companion pet samples, according to one or more embodiments.

FIG. 6 illustrates an exemplary process for using companion pet DNA information to predict a familial relationship of a companion pet, according to one or more embodiments.

FIG. 7 depicts an exemplary environment that may be utilized with techniques presented herein, according to one or more embodiments.

FIG. 8 depicts an example of a computing device that may execute the techniques described herein, according to one or more embodiments.

### DETAILED DESCRIPTION OF EMBODIMENTS

According to certain aspects of the disclosure, methods and systems are disclosed for predicting a familial relationship of a companion pet. Conventional techniques may not be suitable because conventional techniques do not account for inbreeding of a companion pet. Accordingly, improvements in technology relating to predicting familial relationships of companion pets are needed.

Genetic data, such as DNA fragments, are used to find companion pets that are closely related to one another. An example process may include analyzing long fragments of DNA in genomes of at least two companion pets to determine which DNA fragments are identical. Such identical long fragments of DNA may be referred to as “identical-by-descent” (IBD). However, not all identical DNA fragments may be considered IBD. For example, many purebred dogs, which are the product of inbreeding, have long stretches of identical DNA. A small number of individuals founded the breed by inbreeding, in order to develop certain characteristics. Additionally, since the process is unable to determine whether DNA fragments are IBD or a result of inbreeding, the process cannot assign a named familial relationship between the two companion pets. Furthermore, from a genetic point-of-view, the amount of IBD DNA may be the same for different familial relationships, such as great-grandparent/great-grandchild and uncle/aunt/niece/nephew. Thus, a need exists for determining whether DNA fragments are IBD or a result of inbreeding, as well as assigning named familial relationships based on the IBD DNA.

As will be discussed in more detail below, in various embodiments, systems and methods are described for using a machine-learning model to predict a familial relationship of a companion pet. By training a machine-learning model, e.g., via supervised or semi-supervised learning, to learn associations between companion pet DNA information, such as breed, at least one matching companion pet, and/or at least one familial label, the trained machine-learning model may be usable to predict a familial relationship of a companion pet.

Presented in this description are various aspects of machine learning techniques that may be adapted to predict familial relationships of a companion pet. As will be discussed in more detail below, machine learning techniques adapted to predict familial relationships of a companion pet, may include one or more aspects according to this disclosure, e.g., a particular selection of training data, a particular training process for the machine-learning model, operation of a particular device suitable for use with the trained machine-learning model, operation of the machine-learning model in conjunction with particular data, modification of such particular data by the machine-learning model, etc., and/or other aspects that may be apparent to one of ordinary skill in the art based on this disclosure.

### Relative Finder Platform

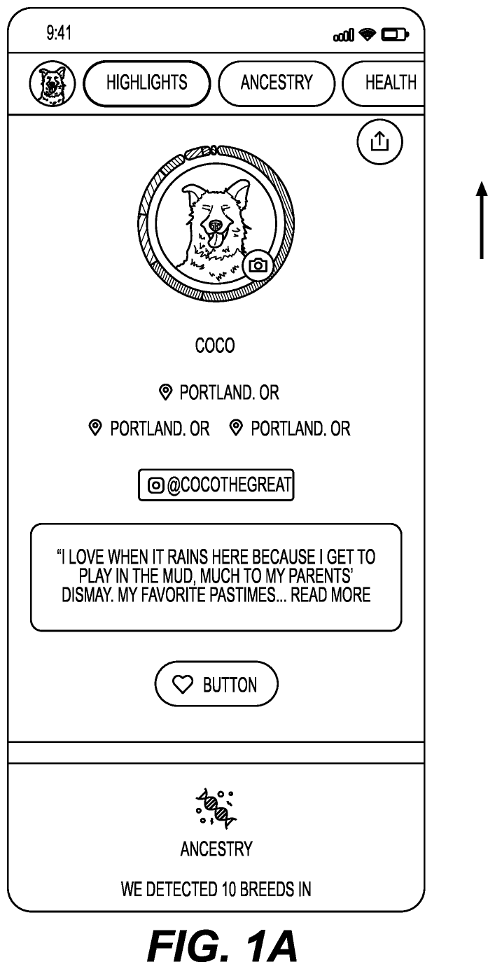

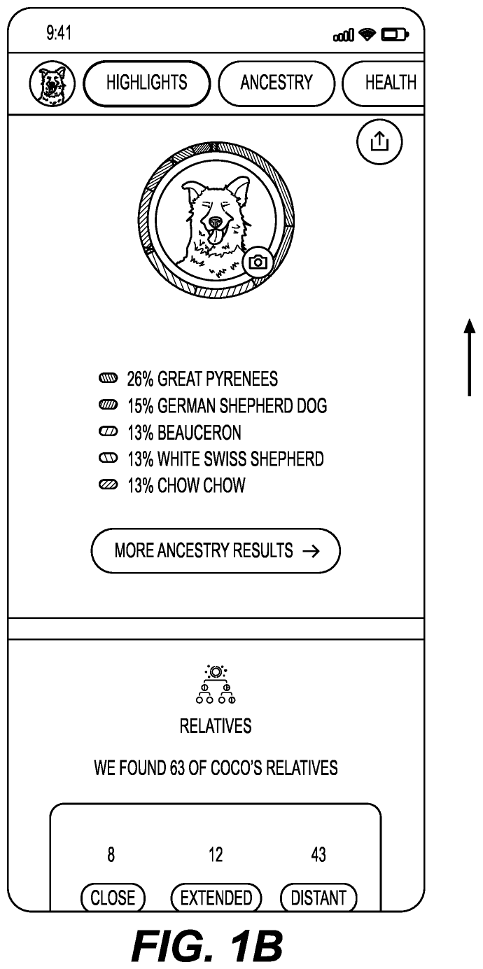

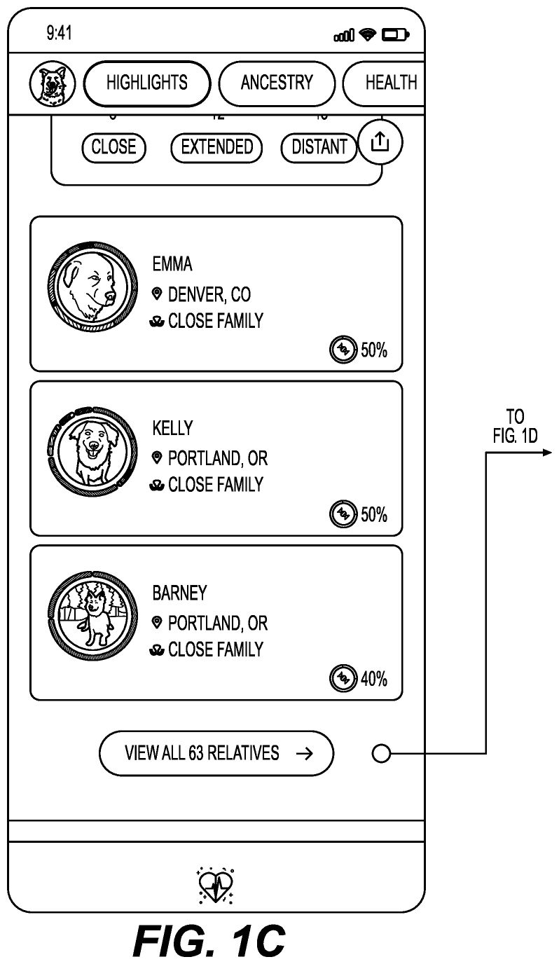

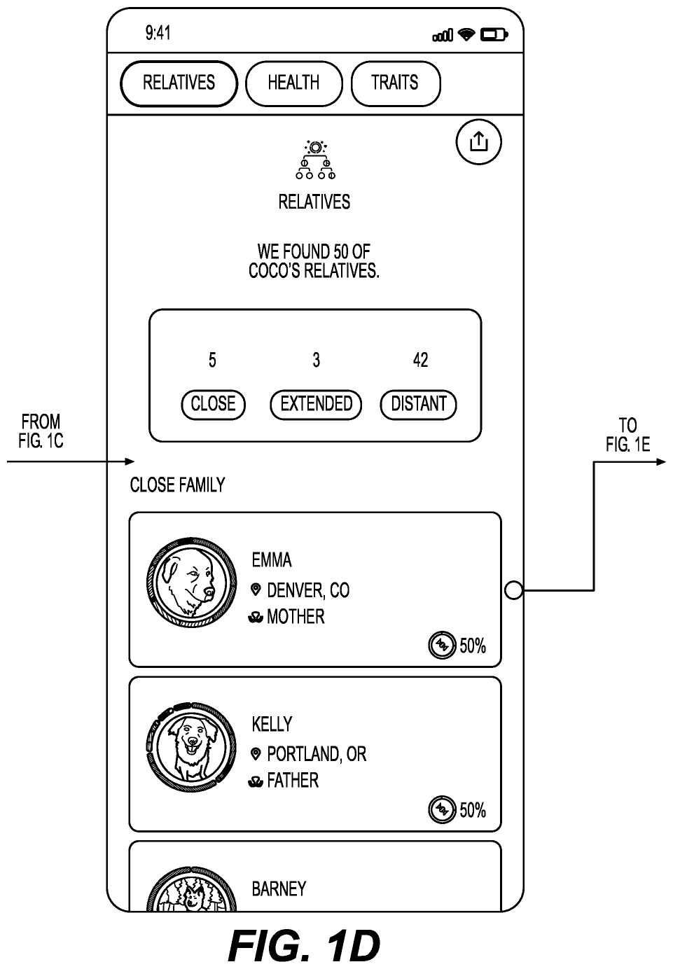

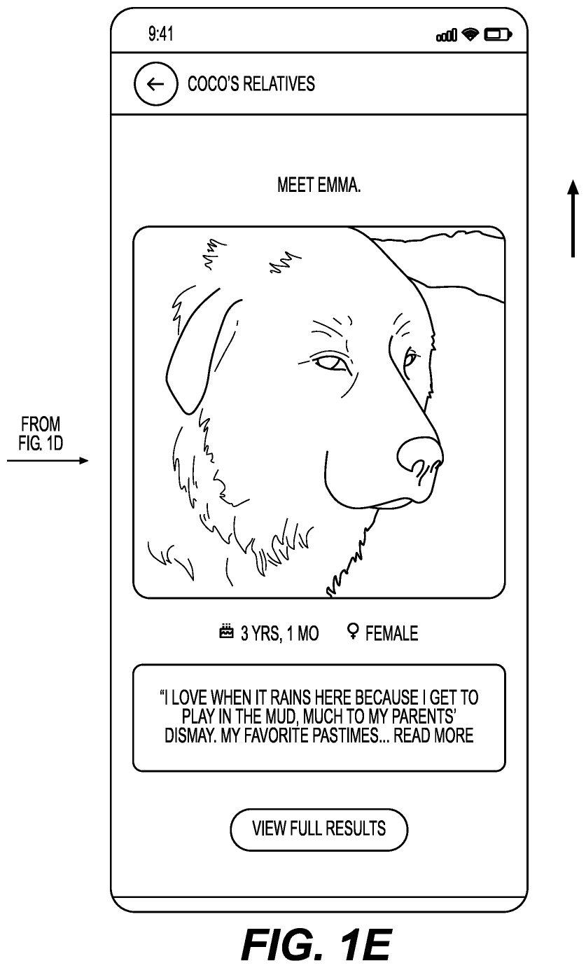

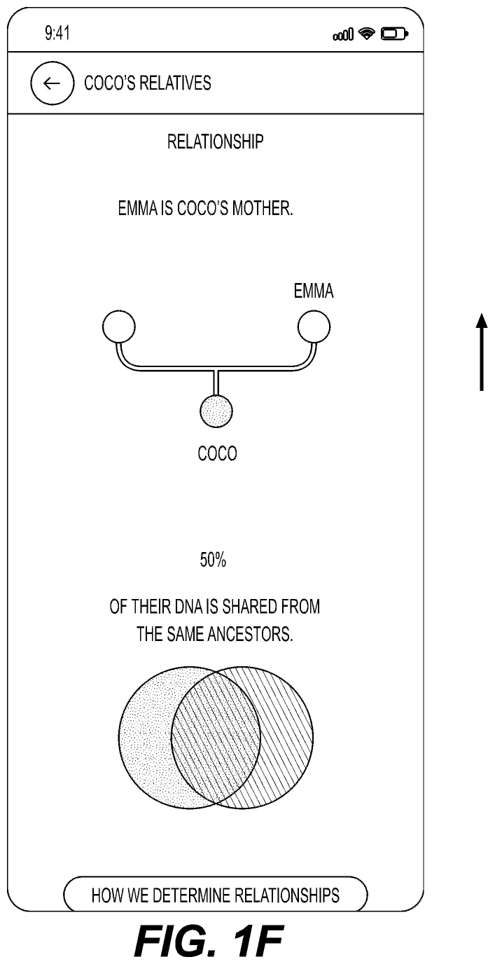

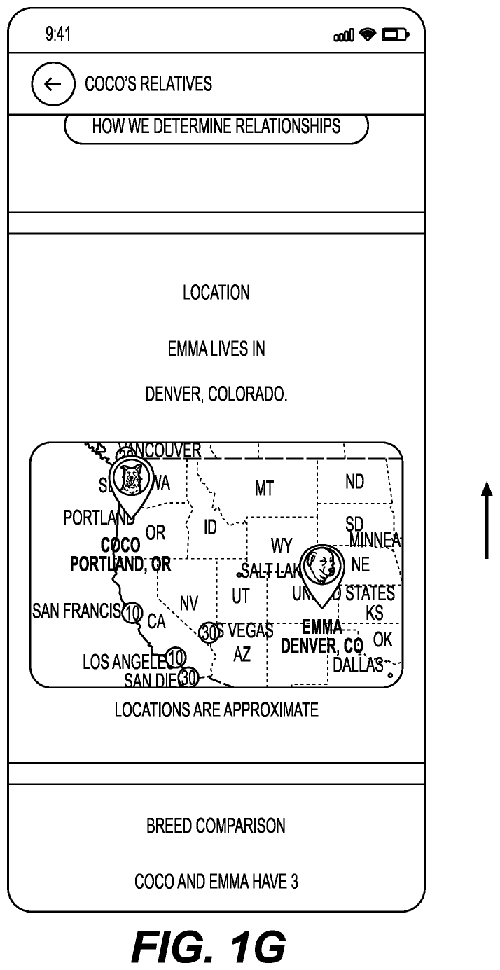

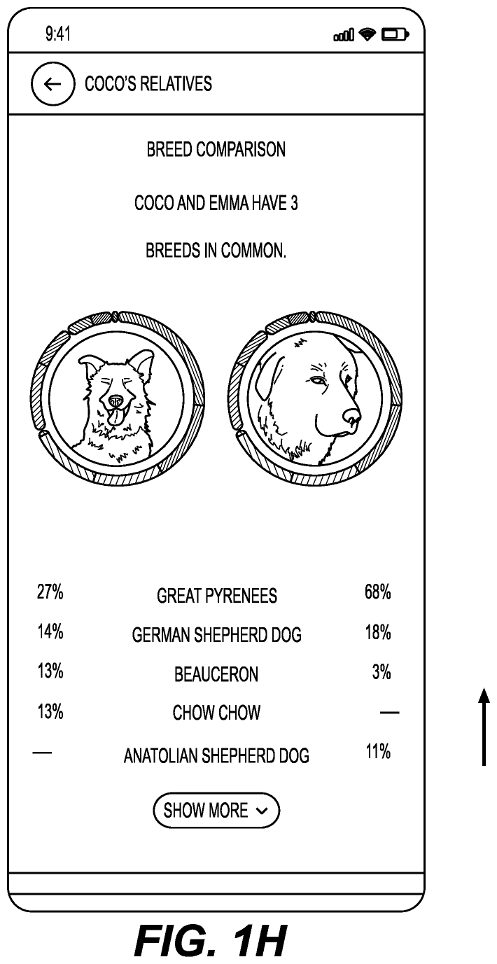

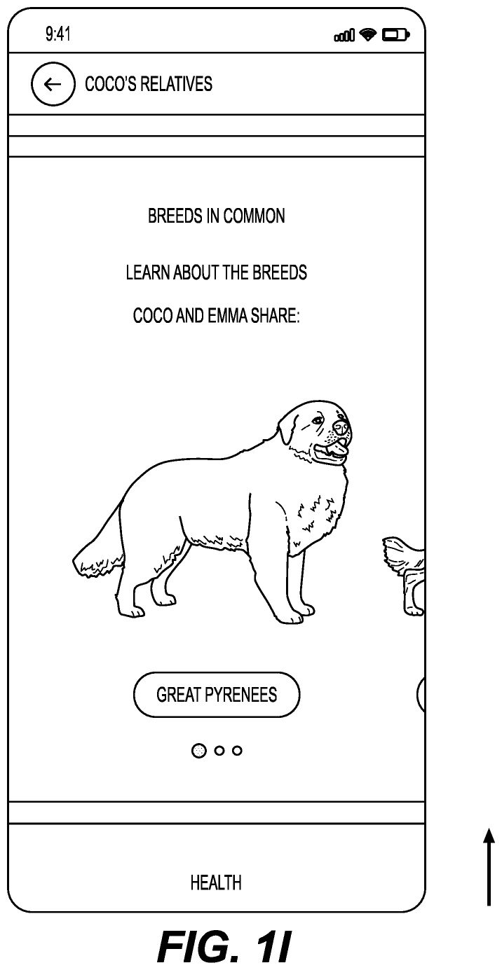

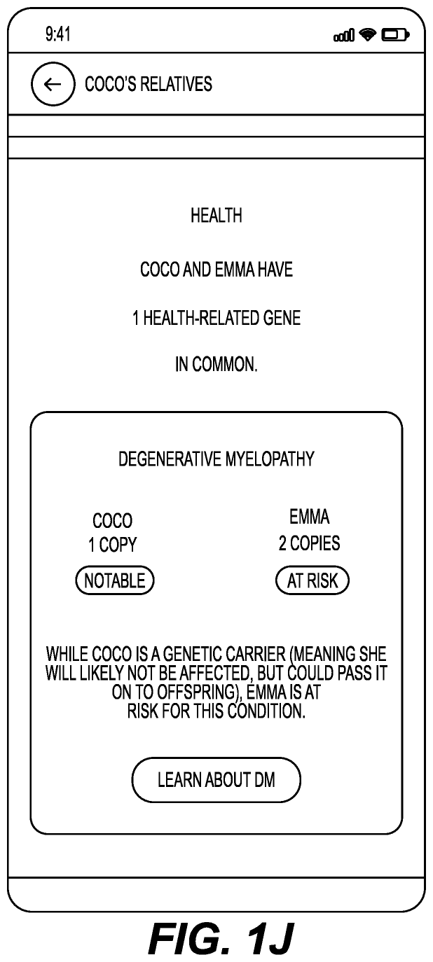

FIGS. 1A-J describe exemplary environments of a platform for predicting a familial relationship of a companion pet, according to one or more embodiments.

FIG. 1A illustrates a “Highlights” page of an exemplary environment of a platform for predicting a familial relationship of a companion pet, according to one or more embodiments. The platform may display a companion pet name (e.g., Coco) and/or an image of the companion pet, along with displaying the companion pet's location (e.g., Portland, Oreg.) and/or a social media account (e.g., @cocothegreat). The platform may further display a “like” button (e.g., a heart button) and/or one or more quotes from the perspective of the companion pet (e.g., “I love it when it rains here because I get to play in the mud, much to my parents' dismay. My favorite pastimes . . . ”). Additionally, a ring of colors may be displayed around the image of the companion pet. The ring of colors (described in further detail below) may correspond to the breed makeup of the companion pet.

FIG. 1B further illustrates a top breeds section of the “Highlights” page of an exemplary environment of a platform for predicting a familial relationship of a companion pet, according to one or more embodiments.

The top breeds section of the “Highlights” page may be accessed by scrolling down (e.g., by swiping up) from the section illustrated in FIG. 1A. In the top breeds section, the platform may further display a breakdown of the breed makeup of the companion pet. For example, in FIG. 1B, the companion pet includes the following breed makeup: Great Pyrenees (26%), German Shepherd Dog (15%), Beauceron (13%), White Swiss Shepherd (13%), and Chow (13%). Additionally, the platform may only display the breeds that reach or exceed a proportion threshold. For example, the proportion threshold may be 6.25%. A machine-learning model may determine that the companion pet includes eight different breeds. However, the machine-learning model may determine that three of the eight different breeds each make up less than 6.25% of the companion pet. As a result, the platform may only display the five breeds that surpass the 6.25% threshold. The platform may further display a ring around the image of the companion pet, where the ring presents a visual indication of the breed makeup of the companion pet. For example, in FIG. 1B, a specific color has been assigned to the Great Pyrenees breed. Since the companion pet is 26% Great Pyrenees, 26% of the ring includes the specific Great Pyrenees color. Additionally, the ring may present a complete visual breakdown of all of the breeds that make up the companion pet, even if all of the breeds do not meet the previously described threshold.

The platform may also display the total number of relatives of the companion pet. The total number of relatives may be determined using the machine-learning model for predicting familial relationships, which is described in more detail below. In addition to displaying the total number of relatives, the platform may display a breakdown of the total number of relatives. More specifically, the platform may display the number of close relatives, the number of extended relatives, and/or the number of distant relatives. For example, in FIG. 1B, the platform indicates that the companion pet has 8 close relatives, 12 extended relatives, and 43 distant relatives.

FIG. 1C further illustrates a close family section of the “Highlights” page of an exemplary environment of a platform for predicting a familial relationship of a companion pet, according to one or more embodiments.

The close family section of the “Highlights” page may be accessed by scrolling further down (e.g., by swiping up) from the section illustrated in FIG. 1B. In the close family section, the platform may display a subset of profile thumbnails of the matching companion pets that are relatives of the companion pet. For example, the platform may display the profile thumbnails of matching companion pets that are only close relatives of the companion pet. Each profile thumbnail may include an image, a name, a ring, a location, a type of relationship, and/or a commonality ratio. For example, the image may be a photo of the matching companion pet, where the ring may be around the exterior of the image. The ring may present a visual indication of the breed makeup of the matching companion pet. The name (e.g., Emma) and the location (e.g., Denver, Colo.) may also be included in the profile thumbnail. The platform may also display the type of relationship (e.g., Close Family) between the matching companion pet and the companion pet. For example, the platform may display whether the matching companion pet is a close relative, an extended relative, or a distant relative of the companion pet. The platform may further display a commonality ratio, which indicates a proportion of DNA fragments that the companion pet and the matching companion pet share. For example, the commonality ratio may be expressed as a percentage (e.g., 50%). The order by which the matching companion pets are listed in the close family section may be based on the commonality ratio. Additionally, the platform may present the option for the user to view all of the matching companion pets, not just the subset of the matching companion pets (e.g., “View All 63 Relatives”).

FIG. 1D illustrates a “Relatives” page of an exemplary environment of a platform for predicting a familial relationship of a companion pet, according to one or more embodiments. For example, in response to the user selecting the “View All 63 Relatives” button on the “Highlights” page, the platform may display all of the profile thumbnails corresponding to the matching companion pets. The platform may display the familial label (e.g., mother) for some and/or all of the profile thumbnails.

FIG. 1E illustrates a matching companion pet's detailed profile page of an exemplary environment of a platform for predicting a familial relationship of a companion pet, according to one or more embodiments.

For example, in response to a user selecting a profile thumbnail, the platform may display a detailed profile page corresponding to the matching companion pet. The detailed profile page may include a name of the matching companion pet (e.g., Emma), an image of the matching companion pet, an age of the companion pet (e.g., 3 yrs, 1 mo), a gender of the companion pet (e.g., Female), and/or one or more quotes from the perspective of the companion pet (e.g., “I love it when it rains here because I get to play in the mud, much to my parents' dismay. My favorite pastimes . . . ”).

FIG. 1F further illustrates a relationship section of the matching companion pet's detailed profile page of an exemplary environment of a platform for predicting a familial relationship of a companion pet, according to one or more embodiments.

The relation section of the matching companion pet's detailed profile page may be accessed by scrolling down (e.g., by swiping up) from the section illustrated in FIG. 1E. The platform may further display a familial relationship, information graphics, and/or a commonality ratio. The platform may display the familial relationship between the companion pet and the corresponding matching companion pet. The informational graphics may visually indicate the familial relationship. Additionally, the commonality ratio may indicate a proportion of DNA fragments that the companion pet and the matching companion pet share. The commonality ratio may further be indicated using an informational graphic, such as a Venn diagram. The familial relationship may be predicted by a machine-learning model, and such a process is described below.

FIG. 1G further illustrates a location section of the matching companion pet's detailed profile page of an exemplary environment of a platform for predicting a familial relationship of a companion pet, according to one or more embodiments. The location section of the matching companion pet's detailed profile page may be accessed by scrolling further down (e.g., by swiping up) from the section illustrated in FIG. 1F. The platform may further display the location of where the matching companion lives (e.g., “Emma lives in Denver, Colo.”), along with a detailed map view that indicates where both the companion pet and the matching companion pet live.

FIG. 1H illustrates a breed comparison section of the matching companion pet's detailed profile page of an exemplary environment of a platform for predicting a familial relationship of a companion pet, according to one or more embodiments.

The breed comparison section of the matching companion pet's detailed profile page may be accessed by scrolling further down (e.g., by swiping up) from the section illustrated in FIG. 1G. The platform may display a detailed comparison of the DNA fragment's similarities overlap between the companion pet and the matching companion pet. The detailed comparison may include a side-by-side comparison of the companion pet and the matching companion pet, including a list of all of the breeds shared by at least one of the companion pet or the matching companion pet. The side-by-side comparison may also include side-by-side images of the companion pet and the matching companion pet, as well as a corresponding ring around each of the images. The side-by-side comparison may further include a ratio (e.g., a percentage) of how much of each of the listed breeds are included in the companion pet and the matching companion pet. The platform may further display the number of breeds that the companion pet and the matching companion pet have in common (e.g., “Coco and Emma have 3 breeds in common.”).

FIG. 1I illustrates a common breed section of the matching companion pet's detailed profile page of an exemplary environment of a platform for predicting a familial relationship of a companion pet, according to one or more embodiments.

The common breed section of the matching companion pet's detailed profile page may be accessed by scrolling further down (e.g., by swiping up) from the section illustrated in FIG. 1H. The platform may display a list of the companion pet breeds that the companion pet and the matching companion pet have in common. The list may be provided by displaying a graphical image corresponding to each of the companion pet breeds. Additionally, the platform may display a section, or a link, that allows a user to get additional information about each of the shared breeds (e.g., “Great Pyrenees”).

FIG. 1J illustrates a health section of the matching companion pet's detailed profile page of an exemplary environment of a platform for predicting a familial relationship of a companion pet, according to one or more embodiments.

The health section of the matching companion pet's detailed profile page may be accessed by scrolling further down (e.g., by swiping up) from the section illustrated in FIG. 1I. The health section may include information regarding health-related genes that the companion pet and the matching companion pet have in common. The platform may indicate a health-related gene name, a status of the health-related gene, a number of copies of the health-related gene, and/or an opportunity for the user to learn more about the health-related gene. For example, the health-related gene name may be “Degenerative Myelopathy,” and the platform may display how many copies of “Degenerative Myelopathy” are carried by the companion pet and/or the matching companion pet. The platform may further indicate a status of whether the companion pet and/or the matching companion pet are “At Risk” for the health-related gene or the health-related gene is “Notable.” The platform may also provide an opportunity, such as a link, to more information regarding “Degenerative Myelopathy” (e.g., “Learn about DM”).

### Training a Machine-Learning Model to Predict a Familial Relationship

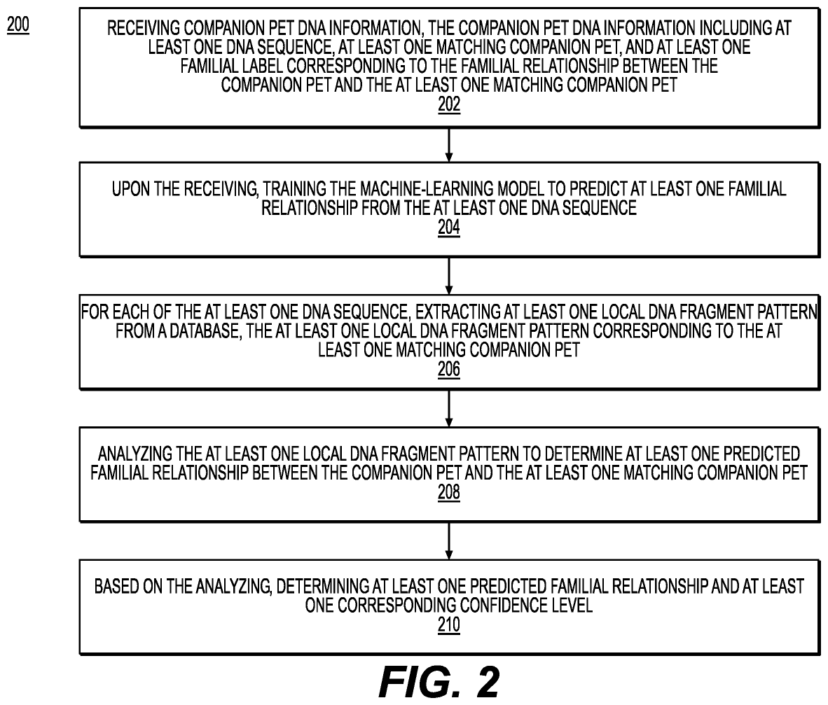

FIG. 2 illustrates an exemplary process for training a machine-learning model to predict a familial relationship of a companion pet, according to one or more embodiments. First, the method may include receiving companion pet DNA information (Step 202). The companion pet DNA information may comprise at least one DNA sequence, at least one matching companion pet, and at least one familial label corresponding to the familial relationship between the companion pet and the at least one matching companion pet. In some embodiments, the companion pet DNA information may further comprise at least one companion pet breed label (e.g., schnauzer, Doberman, etc.). Additionally, for example, the companion pet DNA information may further include whether one or both sets of chromosomes match the at least one matching companion pet, as well as a number of DNA fragments that match the at least one matching companion pet. Furthermore, the at least one familial label may include at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter.

Upon receiving the companion pet DNA information, the method may include training the machine-learning model to predict at least one familial relationship from the at least one DNA sequence (Step 204). Such training is further described in detail below with respect to Steps 206, 208, and 210. In one embodiment, a decision tree model for multi-label classification may be used as the machine-learning model (e.g., a gradient boosting in a decision tree for multi-label classification). However, the machine-learning model contemplated in the current disclosure is not limited to a decision tree model, and may be any model configured to solve the classification problem presented in the current disclosure.

The training may include, for each of the at least one DNA sequence, extracting at least one local DNA fragment pattern from a database, the at least one local DNA fragment pattern corresponding to the matching companion pet (Step 206). The DNA sequence may be input into a database of the DNA sequences of companion pets, where the database may include hundreds, thousands, or millions of entries of DNA sequences of companion pets. Upon receiving the DNA sequence, the database may search for and output at least one local DNA fragment pattern, which may include one or more local matching chromosome segments. The local matching chromosome segments may be an exact match to one or more chromosome segments of the input DNA sequence. The database may provide filtering options when it receives the DNA sequence, such as providing the option of filtering by the length of the local match. For example, it may be beneficial to filter by a local match length of 18 centimorgans, in order to filter out short identical segments, which may be a result of inbreeding in the majority of companion pet breeds. The resulting local chromosome segments may be stitched together in order to account for potential DNA genotyping or phasing errors. The stitched together local chromosome segments may then be aggregated across all chromosomes.

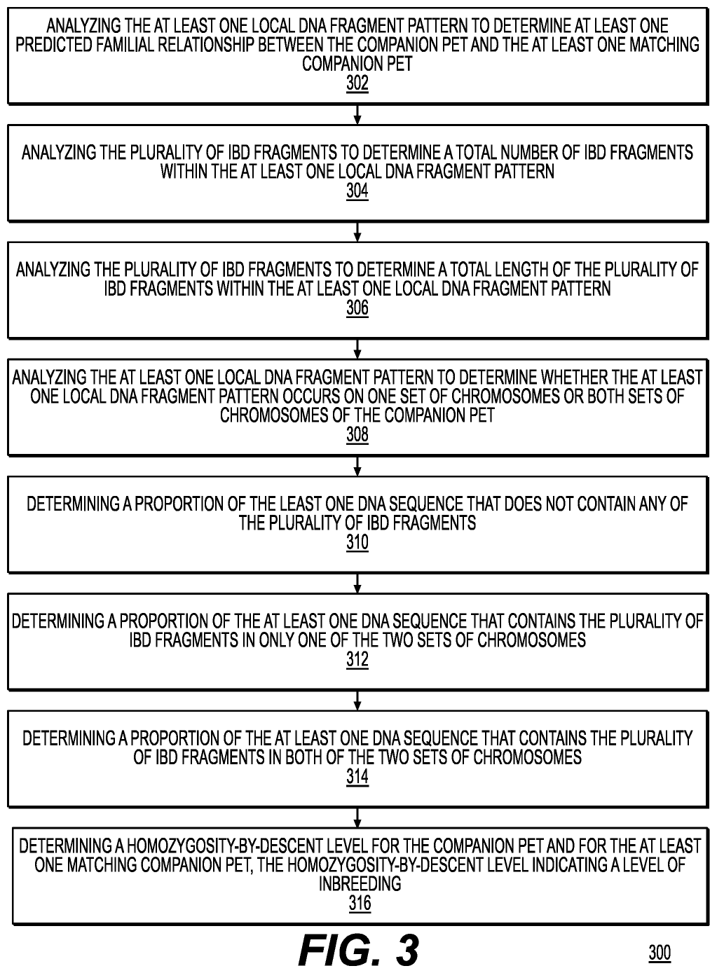

The training may further include analyzing the at least one local DNA fragment pattern to determine at least one predicted familial relationship between the companion pet and the matching companion pet (Step 208). For example, the at least one predicted familial relationship may include at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter. FIG. 3 (below) further describes the analyzing.

The training may also include, based on the analyzing, determining at least one predicted familial relationship and at least one corresponding confidence level (Step 210). Additionally, the method may include determining a match length, where the match length corresponds to the number of fragments of the at least one local DNA fragment pattern. The method may further include storing the at least one predicted familial relationship and/or at least one corresponding confidence level in a database of results. The method may also include displaying the at least one predicted familial relationship, the at least one corresponding confidence level, and/or the match length to a user via a user interface. FIGS. 1C and 1D provide example user interfaces of displaying two predicted familial relationships (e.g., “Mother” and “Father”).

Although FIG. 2 shows example blocks of exemplary method 200, in some implementations, the exemplary method 200 may include additional blocks, fewer blocks, different blocks, or differently arranged blocks than those depicted in FIG. 2. Additionally, or alternatively, two or more of the blocks of the exemplary method 200 may be performed in parallel.

FIG. 3 illustrates an exemplary method 300 for training a machine-learning model to analyze the at least one local DNA fragment pattern, in order to determine at least one predicted familial relationship between the companion pet and the at least one matching companion pet. Notably, method 300 of FIG. 3 corresponds to Step 208 of FIG. 2.

As mentioned above, the method may include analyzing the at least one local DNA fragment pattern to determine at least one predicted familial relationship between the companion pet and the at least one matching companion pet (Step 302). Details or sub-steps of this analyzing step will be discussed in further detail with reference to Steps 304-314 below. Notably, one or more metrics determined in Steps 304-314 may be used in training the machine-learning model.

The at least one local DNA fragment pattern may include a plurality of IBD (identical-by-descent) fragments. The IBD fragments may be long fragments of DNA in a sequence that are exactly the same between a companion pet and a matching companion pet. Additionally, not all of the DNA fragments that are the same between two companion pets may be considered IBD. For example, purebred dogs may have long stretches of identical DNA because the purebred dogs were inbred.

The analyzing may further include analyzing the plurality of IBD fragments to determine a total number of IBD fragments within the at least one local DNA fragment pattern (Step 304).

The analyzing may further include analyzing the plurality of IBD fragments to determine a total length of the plurality of IBD fragments within the at least one local DNA fragment pattern (Step 306). Additionally, the total length of the plurality of IBD fragments may be measured in centimorgans.

The analyzing may further include analyzing the at least one local DNA fragment pattern to determine whether the at least one local DNA fragment pattern occurs on one set of chromosomes or both sets of chromosomes of the companion pet (Step 308).

The analyzing may further include determining a proportion of the at least one DNA sequence that does not contain any of the plurality of IBD fragments (Step 310). The analyzing may further include determining a proportion of the at least one DNA sequence that contains the plurality of IBD fragments in only one of two sets of chromosomes (Step 312). The analyzing may further include determining a proportion of the at least one DNA sequence that contains the plurality of IBD fragments in both of the two sets of chromosomes (Step 314).

The previously described analysis (e.g., the various metrics determined during the analysis) may assist in evaluating the type of familial relationship between the companion pet and the matching companion pet, by providing the metrics to the machine-learning model as features. For example, determining a proportion (referred to as IBD0) of the at least one DNA sequence that does not contain any of the plurality of IBD fragments may be useful in determining if there is a possibility of a familial relationship. If the IBD0 proportion is very high, there may be a small likelihood of a familial relationship. By way of further example, two half-siblings (e.g., share one parent) inherit one set of chromosomes from the same parent. As a result, IBD matching between the two half-siblings will only occur on the chromosome set that was inherited from the same parent. Such a situation (referred to as IBD1) may be established by determining the proportion of the DNA sequence that has IBD fragments for only one of the two sets of chromosomes in both the companion pet and the matching companion pet. By way of another example, full-siblings (e.g., share both parents) will share IBD matching on both sets of chromosomes, since both sets have common ancestors. Such a situation (referred to as IBD2) may be established by determining the proportion of the DNA sequence that has IBD for both sets of chromosomes in both the companion pet and the matching companion pet. Table 1 (shown below) provides further illustrations of such situations.

TABLE 1
IBD Matching

| Relationship | IBD0 | IBD1 | IBD2 |
|---|---|---|---|
| Parent/offspring | 50% | 50% | 0% |
| Full-sibling | 50% | 25% | 25% |
| Half-sibling | 75% | 25% | 0% |
| Uncle/aunt/niece/nephew | 75% | 12.5% | 12.5% |

The method may also include determining a homozygosity-by-descent (HBD) level for the companion pet and for the at least one matching companion pet, the homozygosity-by-descent level indicating a level of inbreeding (Step 316). HBD is informative about the levels of inbreeding in the family tree of the companion pet. Higher levels of HBD are expected for purebred animals versus outbred animals. For example, for every companion pet and matching companion pet pair, the HBD for the companion pet and the HBD for the matching companion pet are two additional metrics for the machine-learning model's overall analysis. In other words, in addition to the metrics determined in Steps 304-314, the HBDs for the companion pet and the matching companion pet may also be used in training the machine-learning model.

In an embodiment, in order to determine a model accuracy, a cross-validation technique may be utilized. For instance, a subset of truth familial labels may be masked from the training set (i.e., training panel), and those labels may be estimated based on the machine-learning model obtained from the remainder of the training set.

Although FIG. 3 shows example blocks of exemplary method 300, in some implementations, the exemplary method 300 may include additional blocks, fewer blocks, different blocks, or differently arranged blocks than those depicted in FIG. 3. Additionally, or alternatively, two or more of the blocks of the exemplary method 300 may be performed in parallel.

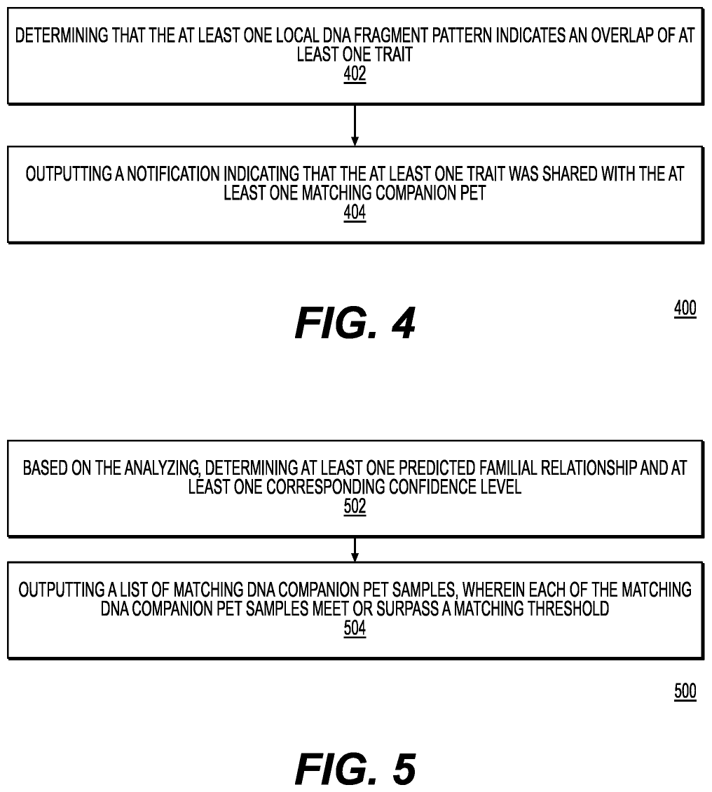

FIG. 4 further illustrates a method 400 for training the machine-learning model to predict at least one shared trait between a companion pet and a matching companion pet, according to one or more embodiments.

The method may include determining that the at least one local DNA fragment pattern indicates an overlap of at least one trait (Step 402). For example, the at least one trait may be breed-specific. Example traits may include eye color, coat color, and/or a health-related gene. Upon determining the overlap of the at least one trait, the method may further include outputting a notification indicating that the at least one trait was shared with the at least one matching companion pet (Step 404).

Although FIG. 4 shows example blocks of exemplary method 400, in some implementations, the exemplary method 400 may include additional blocks, fewer blocks, different blocks, or differently arranged blocks than those depicted in FIG. 4. Additionally, or alternatively, two or more of the blocks of the exemplary method 400 may be performed in parallel.

FIG. 5 further illustrates a method 500 for training the machine-learning model to output a list of matching DNA companion pet samples, according to one or more embodiments. Notably, method 500 may complement method 200 of FIG. 2, with Step 502 corresponding to Step 210 of FIG. 2.

As discussed above in reference to FIG. 2, the method may include, based on the analyzing, determining at least one predicted familial relationship and at least one corresponding confidence level (Step 502). Based on the determining performed by the machine-learning model, the method may further include outputting a list of matching DNA companion pet samples, wherein each of the matching DNA companion pet samples meet or surpass a matching threshold (Step 504). For example, if the database returns more than one local DNA fragment pattern, the list may display all of the local DNA fragment patterns. Additionally, only a subset of the local DNA fragment patterns may be displayed, where the displayed local DNA fragment patterns meet or surpass a threshold. The threshold may include a maximum number of entries to display, such as 50, or the threshold may include a cutoff DNA proportion match. The cutoff DNA proportion match may be calculated by determining the percentage of local DNA fragments of the database that match all of the DNA fragments of the companion pet. For example, the threshold may include a cutoff DNA proportion of 6.25%.

Although FIG. 5 shows example blocks of exemplary method 500, in some implementations, the exemplary method 500 may include additional blocks, fewer blocks, different blocks, or differently arranged blocks than those depicted in FIG. 5. Additionally, or alternatively, two or more of the blocks of the exemplary method 500 may be performed in parallel.

### Using the Trained Machine-Learning Model to Predict a Familial Relationship

An exemplary process for predicting a familial relationship of a companion pet, according to one or more embodiments, is discussed below (e.g., by utilizing a trained machine-learning model such as a machine-learning model trained according to one or more embodiments discussed above).

The method may include receiving companion pet DNA information. The information may include at least one DNA sequence. A machine-learning model, which may have been previously trained, may receive the companion pet DNA information.

Upon receiving the companion pet DNA information, the method may include predicting at least one familial relationship from the at least one DNA sequence using the trained machine-learning model. The predicting may be further described in detail below.

For each of the at least one DNA sequence, the method may include extracting at least one local DNA fragment pattern from a database, the at least one local DNA fragment pattern corresponding to at least one matching companion pet. The DNA sequence may be input into a DNA sequence database of companion pets, where the DNA sequence database may include hundreds, thousands, or millions of entries of DNA sequences of companion pets. Upon receiving the DNA sequence, the DNA sequence database may search for and then output at least one local DNA fragment pattern, which may include one or more local matching chromosome segments. Such local chromosome segments may be an exact match to one or more chromosome segments of the input DNA sequence. The database may provide filtering options when it receives the DNA sequence, such as providing the option of filtering by the length of the local match. For example, it may be beneficial to filter by a local match length of 18 centimorgans, in order to filter out short identical segments, which may be a result of inbreeding. The resulting local chromosome segments may be stitched together in order to account for potential DNA genotyping or phasing errors. The stitched together local chromosome segments may then be aggregated across all chromosomes.

The method may include analyzing the at least one local DNA fragment pattern to determine at least one predicted familial relationship between the companion pet and the at least one matching companion pet. For example, the at least one predicted familial relationship may include at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter. The analyzing is described in further detail below.

The at least one local DNA fragment pattern may include a plurality of IBD fragments. Additionally, not all of the DNA fragments that are the same between two companion pets may be considered IBD. For example, purebred pets may have long stretches of identical DNA the breed may have been inbred.

The analyzing may further include analyzing the plurality of IBD fragments to determine a total number of IBD fragments within the at least one local DNA fragment pattern.

The analyzing may further include analyzing the plurality of IBD fragments to determine a total length of the plurality of IBD fragments within the at least one local DNA fragment pattern. Additionally, the total length of the plurality of IBD fragments may be measured in centimorgans.

The analyzing may further include analyzing the at least one local DNA fragment pattern to determine whether the at least one local DNA fragment pattern occurs on one set of chromosomes or both sets of chromosomes of the companion pet.

The analyzing may further include determining a proportion of the at least one DNA sequence that does not contain any of the plurality of IBD fragments. The analyzing may further include determining a proportion of the at least one DNA sequence that contains the plurality of IBD fragments in only one of two sets of chromosomes. The analyzing may further include determining a proportion of the at least one DNA sequence that contains the plurality of IBD fragments in both sets of chromosomes.

The previously described analysis (e.g., the various metrics determined during the analysis) may assist in evaluating the type of familial relationship between the companion pet and the matching companion pet, by providing the determined metrics to the trained machine learning model as features. For example, determining a proportion (referred to as IBD0) of the at least one DNA sequence that does not contain any of the plurality of IBD fragments may be useful in determining if there is a possibility of a familial relationship. If the IBD0 proportion is very high, there may be a small likelihood of a familial relationship. By way of further example, two half-siblings (e.g., share one parent) inherit one set of chromosomes from the same parent. As a result, IBD matching between the two half-siblings will only occur on the chromosome set that was inherited from the same parent. Such a situation (referred to as IBD1) may be established by determining the proportion of the DNA sequence that has IBD for only one of the two sets of chromosomes in both the companion pet and the matching companion pet. By way of another example, full-siblings (e.g., share both parents) will share IBD matching on both sets of chromosomes, since both sets have common ancestors. Such a situation (referred to as IBD2) may be established by determining the proportion of the DNA sequence that has IBD for both sets of chromosomes in both the companion pet and the matching companion pet.

The method may further include determining a homozygosity-by-descent level for the companion pet and for the at least one matching companion pet, the homozygosity-by-descent level indicating a level of inbreeding. HBD is informative about the levels of inbreeding in the family tree of the companion pet. Higher levels of HBD are expected for purebred animals versus outbred animals. For example, for every companion pet and matching companion pet pair, the HBD for the companion pet and the HBD for the matching companion pet are two additional metrics for the machine-learning model's overall analysis. In other words, in addition to the metrics determined previously (e.g., based on analyzing the at least one local DNA fragment pattern), the HBDs for the companion pet and the matching companion pet may also be provided to the trained machine-learning model to predict a familial relationship.

The method may also include determining that the at least one local DNA fragment pattern indicates an overlap of at least one trait. For example, the at least one trait may be breed-specific. Example traits may include eye color, coat color, and/or a health-related gene. Upon determining the overlap of the at least one trait, the method may include outputting a notification indicating that the at least one trait was shared with the at least one matching companion pet.

Based on the analysis performed by the machine-learning model, at least one predicted familial relationship and at least one corresponding confidence level between the companion pet and the matching companion pet may be determined. Additionally, the method may further include determining a match length, where the match length corresponds to the number of fragments of the at least one local DNA fragment pattern. The method may further include storing the at least one predicted familial relationship and/or at least one corresponding confidence level in a database of results. The method may also include displaying the at least one predicted familial relationship, the at least one corresponding confidence level, and/or the match length to a user via a user interface. FIGS. 1C and 1D provide example user interfaces of displaying two predicted familial relationships (e.g., “Mother” and “Father”).

Additionally, based on the determining performed by the machine-learning model, the method may also further include outputting a list of matching DNA companion pet samples, wherein each of the matching DNA companion pet samples meet or surpass a matching threshold. For example, if the database returns more than one local DNA fragment pattern, the list may display all of the local DNA fragment patterns. Additionally, only a subset of the local DNA fragment patterns may be displayed, where the displayed local DNA fragment patterns meet or surpass a threshold. The threshold may include a maximum number of entries to display, such as 50, or the threshold may include a cutoff DNA proportion match. The proportion may be calculated by determining the percentage of local DNA fragments of the database companion pet that match all of the DNA fragments in the companion pet. For example, the threshold may include a cutoff DNA proportion of 6.25%.

### Exemplary Process for Predicting a Familial Relationship of a Companion Pet

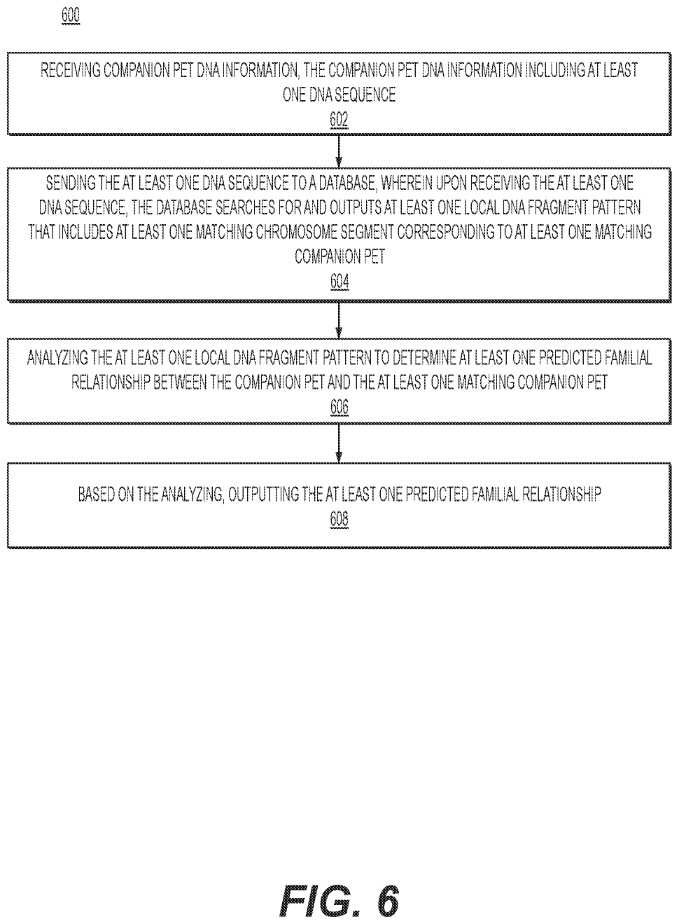

FIG. 6 illustrates an exemplary process for using companion pet DNA information to predict a familial relationship of a companion pet, according to one or more embodiments.

The method may include receiving companion pet DNA information, the companion pet DNA information including at least one DNA sequence (Step 602).

The method may include sending the at least one DNA sequence to a database, wherein upon receiving the at least one DNA sequence, the database searches for and outputs at least one local DNA fragment pattern that includes at least one matching chromosome segment corresponding to at least one matching companion pet (Step 604). The DNA sequence may be input into a database of the DNA sequences of companion pets, where the database may include hundreds, thousands, or millions of entries of DNA sequences of companion pets. Upon receiving the DNA sequence, the database may search for and output at least one local DNA fragment pattern, which may include one or more local matching chromosome segments. The local matching chromosome segments may be an exact match to one or more chromosome segments of the input DNA sequence. The database may provide filtering options when it receives the DNA sequence, such as providing the option of filtering by the length of the local match. For example, it may be beneficial to filter by a local match length of 18 centimorgans, in order to filter out short identical segments, which may be a result of inbreeding in the majority of companion pet breeds. The resulting local chromosome segments may be stitched together in order to account for potential DNA genotyping or phasing errors. The stitched together local chromosome segments may then be aggregated across all chromosomes.

The method may include analyzing the at least one local DNA fragment pattern to determine at least one predicted familial relationship between the companion pet and the at least one matching companion pet (Step 606). The at least one local DNA fragment pattern may include a plurality of IBD (identical-by-descent) fragments.

The method may include, based on the analyzing, outputting the at least one predicted familial relationship (Step 608). The at least one predicted familial relationship may include at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter. The method may also include determining at least one corresponding confidence level. Additionally, the method may include determining a match length, where the match length corresponds to the number of fragments of the at least one local DNA fragment pattern. The method may further include storing the at least one predicted familial relationship and/or at least one corresponding confidence level in a database of results. The method may also include displaying the at least one predicted familial relationship, the at least one corresponding confidence level, and/or the match length to a user via a user interface. FIGS. 1C and 1D provide example user interfaces of displaying two predicted familial relationships (e.g., “Mother” and “Father”).

In some embodiments, the method may include outputting a list of matching DNA companion pet samples, wherein each of the matching DNA companion pet samples meet or surpass a matching threshold. For example, if the database returns more than one local DNA fragment pattern, the list may display all of the local DNA fragment patterns. Additionally, only a subset of the local DNA fragment patterns may be displayed, where the displayed local DNA fragment patterns meet or surpass a threshold. The threshold may include a maximum number of entries to display, such as 50, or the threshold may include a cutoff DNA proportion match. The cutoff DNA proportion match may be calculated by determining the percentage of local DNA fragments of the database that match all of the DNA fragments of the companion pet. For example, the threshold may include a cutoff DNA proportion of 6.25%.

In some embodiments, the method may include determining that the at least one local DNA fragment pattern indicates an overlap of at least one trait. Example traits may include eye color, coat color, and/or a health-related gene. The method may also include outputting a notification indicating that the at least one trait was shared with the at least one matching companion pet. Additionally, the at least one trait may be breed-specific.

In some embodiments, the method may include determining a homozygosity-by-descent level for the companion pet and for the at least one matching companion pet, the homozygosity-by-descent level indicating a level of inbreeding. HBD may be informative about the levels of inbreeding in the family tree of the companion pet. Higher levels of HBD may be expected for purebred animals versus outbred animals.

In some embodiments, the method may include analyzing the plurality of IBD fragments to determine a total number of IBD fragments within the at least one local DNA fragment pattern.

In some embodiments, the method may include analyzing the plurality of IBD fragments to determine a total length of the plurality of IBD fragments within the at least one local DNA fragment pattern. Additionally, the total length of the plurality of IBD fragments may be measured in centimorgans.

In some embodiments, the method may include analyzing the at least one local DNA fragment pattern to determine whether the at least one local DNA fragment pattern occurs on one set of chromosomes or both sets of chromosomes of the companion pet. In some embodiments, the method may further include determining a proportion of the at least one DNA sequence that does not contain any of the plurality of IBD fragments. The analyzing may further include determining a proportion of the at least one DNA sequence that contains the plurality of IBD fragments in only one of two sets of chromosomes. The analyzing may further include determining a proportion of the at least one DNA sequence that contains the plurality of IBD fragments in both of the two sets of chromosomes.

Although FIG. 6 shows example blocks of exemplary method 600, in some implementations, the exemplary method 600 may include additional blocks, fewer blocks, different blocks, or differently arranged blocks than those depicted in FIG. 6. Additionally, or alternatively, two or more of the blocks of the exemplary method 600 may be performed in parallel.

### Exemplary Environment and Exemplary Device

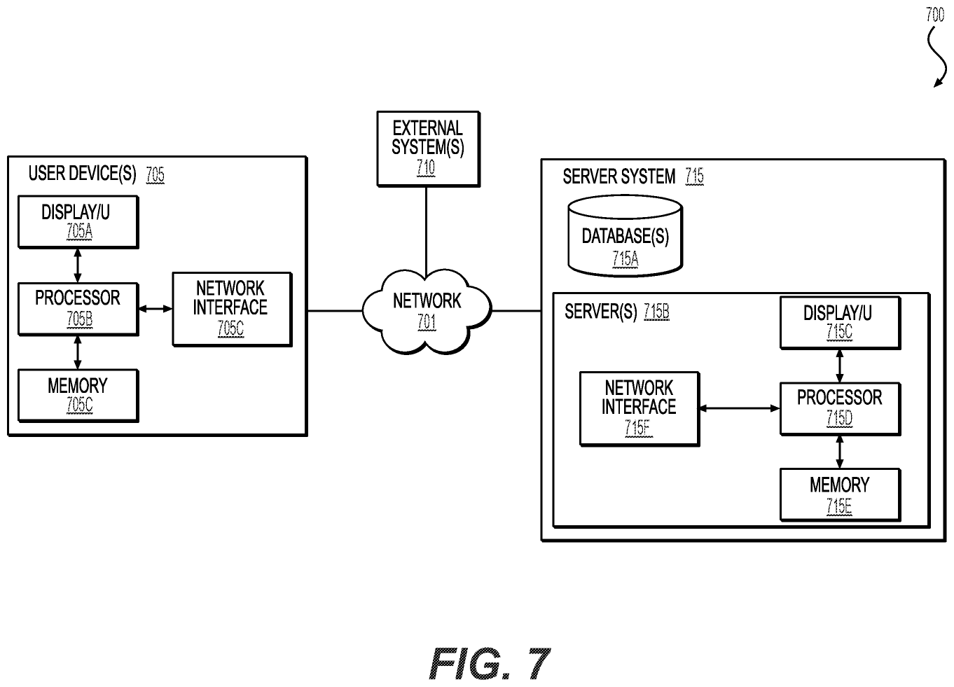

FIG. 7 depicts an exemplary environment 700 that may be utilized with techniques presented herein. One or more user device(s) 705, one or more external system(s) 710, and one or more server system(s) 715 may communicate across a network 701. As will be discussed in further detail below, one or more server system(s) 715 may communicate with one or more of the other components of the environment 700 across network 701. The one or more user device(s) 705 may be associated with a user, e.g., a user associated with one or more of generating, training, or tuning a machine-learning model for predicting a familial relationship of a companion pet.

In some embodiments, the components of the environment 700 are associated with a common entity, e.g., a veterinarian, clinic, animal specialist, research center, or the like. In some embodiments, one or more of the components of the environment is associated with a different entity than another. The systems and devices of the environment 700 may communicate in any arrangement. As will be discussed herein, systems and/or devices of the environment 700 may communicate in order to one or more of generate, train, and/or use a machine-learning model to predict a familial relationship of a companion pet, among other activities.

The user device 705 may be configured to enable the user to access and/or interact with other systems in the environment 700. For example, the user device 705 may be a computer system such as, for example, a desktop computer, a mobile device, a tablet, etc. In some embodiments, the user device 705 may include one or more electronic application(s), e.g., a program, plugin, browser extension, etc., installed on a memory of the user device 705.

The user device 705 may include a display/user interface (UI) 705A, a processor 705B, a memory 705C, and/or a network interface 705D. The user device 705 may execute, by the processor 705B, an operating system (O/S) and at least one electronic application (each stored in memory 705C). The electronic application may be a desktop program, a browser program, a web client, or a mobile application program (which may also be a browser program in a mobile O/S), an applicant specific program, system control software, system monitoring software, software development tools, or the like. For example, environment 700 may extend information on a web client that may be accessed through a web browser. In some embodiments, the electronic application(s) may be associated with one or more of the other components in the environment 700. The application may manage the memory 705C, such as a database, to transmit streaming data to network 701. The display/UI 705A may be a touch screen or a display with other input systems (e.g., mouse, keyboard, etc.) so that the user(s) may interact with the application and/or the O/S. The network interface 705D may be a TCP/IP network interface for, e.g., Ethernet or wireless communications with the network 701. The processor 705B, while executing the application, may generate data and/or receive user inputs from the display/UI 705A and/or receive/transmit messages to the server system 715, and may further perform one or more operations prior to providing an output to the network 701.

External systems 710 may be, for example, one or more third party and/or auxiliary systems that integrate and/or communicate with the server system 715 in performing various document information extraction tasks. External systems 710 may be in communication with other device(s) or system(s) in the environment 700 over the one or more networks 701. For example, external systems 710 may communicate with the server system 715 via API (application programming interface) access over the one or more networks 701, and also communicate with the user device(s) 705 via web browser access over the one or more networks 701.

In various embodiments, the network 701 may be a wide area network (“WAN”), a local area network (“LAN”), a personal area network (“PAN”), or the like. In some embodiments, network 701 includes the Internet, and information and data provided between various systems occurs online. “Online” may mean connecting to or accessing source data or information from a location remote from other devices or networks coupled to the Internet. Alternatively, “online” may refer to connecting or accessing a network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

The server system 715 may include an electronic data system, e.g., a computer-readable memory such as a hard drive, flash drive, disk, etc. In some embodiments, the server system 715 includes and/or interacts with an application programming interface for exchanging data to other systems, e.g., one or more of the other components of the environment.

The server system 715 may include a database 715A and at least one server 715B. The server system 715 may be a computer, system of computers (e.g., rack server(s)), and/or or a cloud service computer system. The server system may store or have access to database 715A (e.g., hosted on a third party server or in memory 715E). The server(s) may include a display/UI 715C, a processor 715D, a memory 715E, and/or a network interface 715F. The display/UI 715C may be a touch screen or a display with other input systems (e.g., mouse, keyboard, etc.) for an operator of the server 7156 to control the functions of the server 7156. The server system 715 may execute, by the processor 715D, an operating system (O/S) and at least one instance of a servlet program (each stored in memory 715E).

The server system 715 may generate, store, train, or use a machine-learning model, configured to predict a familial relationship of a companion pet. The server system 715 may include a machine-learning model and/or instructions associated with the machine-learning model, e.g., instructions for generating a machine-learning model, training the machine-learning model, using the machine-learning model, etc. The server system 715 may include instructions for retrieving DNA information data, e.g., based on the output of the machine-learning model, and/or operating the display 715C to output familial relationship information data, e.g., as adjusted based on the machine-learning model. The server system 715 may include training data, e.g., at least one DNA sequence, at least one matching companion pet, and at least one familial label corresponding to the familial relationship between the companion pet and the at least one matching companion pet.

In some embodiments, a system or device other than the server system 715 is used to generate and/or train the machine-learning model. For example, such a system may include instructions for generating the machine-learning model, the training data and ground truth, and/or instructions for training the machine-learning model. A resulting trained machine-learning model may then be provided to the server system 715.

Generally, a machine-learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some embodiments, a portion of the training data may be withheld during training and/or used to validate the trained machine-learning model, e.g., compare the output of the trained model with the ground truth for that portion of the training data to evaluate an accuracy of the trained model. The training of the machine-learning model may be configured to cause the machine-learning model to learn associations between the DNA information data and the familial relationship data, such that the trained machine-learning model is configured to determine a familial relationship in response to the input DNA information data based on the learned associations.

In various embodiments, the variables of a machine-learning model may be interrelated in any suitable arrangement in order to generate the output. For example, in some embodiments, the machine-learning model may include signal processing architecture that is configured to identify, isolate, and/or extract features, patterns, and/or structure in a text. For example, the machine-learning model may include one or more convolutional neural network (“CNN”) configured to identify features in the document information data, and may include further architecture, e.g., a connected layer, neural network, etc., configured to determine a relationship between the identified features in order to determine a location in the document information data.

Although depicted as separate components in FIG. 7, it should be understood that a component or portion of a component in the environment 700 may, in some embodiments, be integrated with or incorporated into one or more other components. For example, a portion of the display 715C may be integrated into the user device 705 or the like. In some embodiments, operations or aspects of one or more of the components discussed above may be distributed amongst one or more other components. Any suitable arrangement and/or integration of the various systems and devices of the environment 700 may be used.

Further aspects of the machine-learning model and/or how it may be utilized to predict a familial relationship of a companion pet are discussed in further detail in the methods above. In the following methods, various acts may be described as performed or executed by a component from FIG. 7, such as the server system 715, the user device 705, or components thereof. However, it should be understood that in various embodiments, various components of the environment 700 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

In general, any process or operation discussed in this disclosure that is understood to be computer-implementable, such as the processes illustrated in FIGS. 1-6, may be performed by one or more processors of a computer system, such any of the systems or devices in the environment 700 of FIG. 7, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 7. One or more processors of a computer system may be included in a single computing device or distributed among a plurality of computing devices. A memory of the computer system may include the respective memory of each computing device of the plurality of computing devices.

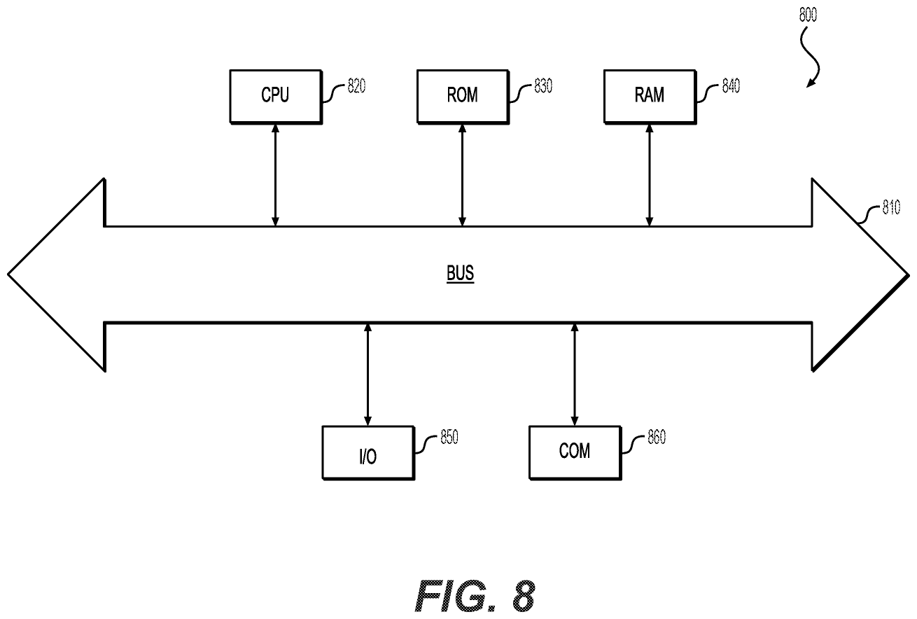

FIG. 8 is a simplified functional block diagram of a computer 800 that may be configured as a device for executing the methods of FIGS. 1-6, according to exemplary embodiments of the present disclosure. For example, device 800 may include a central processing unit (CPU) 820. CPU 820 may be any type of processor device including, for example, any type of special purpose or a general-purpose microprocessor device. As will be appreciated by persons skilled in the relevant art, CPU 820 also may be a single processor in a multi-core/multiprocessor system, such system operating alone, or in a cluster of computing devices operating in a cluster or server farm. CPU 820 may be connected to a data communication infrastructure 810, for example, a bus, message queue, network, or multi-core message-passing scheme.

Device 800 also may include a main memory 840, for example, random access memory (RAM), and also may include a secondary memory 830. Secondary memory 830, e.g., a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage unit may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 830 may include other similar means for allowing computer programs or other instructions to be loaded into device 800. Examples of such means may include a program cartridge and cartridge interface (such as that found in video game devices), a removable memory chip (such as an EPROM, or PROM) and associated socket, and other removable storage units and interfaces, which allow software and data to be transferred from a removable storage unit to device 800.

Device 800 also may include a communications interface (“COM”) 860. Communications interface 860 allows software and data to be transferred between device 800 and external devices. Communications interface 860 may include a modem, a network interface (such as an Ethernet card), a communications port, a PCMCIA slot and card, or the like. Software and data transferred via communications interface 860 may be in the form of signals, which may be electronic, electromagnetic, optical, or other signals capable of being received by communications interface 860. These signals may be provided to communications interface 860 via a communications path of device 800, which may be implemented using, for example, wire or cable, fiber optics, a phone line, a cellular phone link, an RF link or other communications channels.

The hardware elements, operating systems and programming languages of such equipment are conventional in nature, and it is presumed that those skilled in the art are adequately familiar therewith. Device 800 also may include input and output ports 850 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Reference to any particular activity is provided in this disclosure only for convenience and not intended to limit the disclosure. A person of ordinary skill in the art would recognize that the concepts underlying the disclosed devices and methods may be utilized in any suitable activity. The disclosure may be understood with reference to the following description and the appended drawings, wherein like elements are referred to with the same reference numerals.

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the general description and the detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, a term such as “user” or the like generally encompasses a pet parent and/or pet parents. A term such as “pet” or the like generally encompasses a user's pet, where the term may encompass multiple pets. A term such as “provider” or the like generally encompasses a pet care business

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model/system is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine-learning techniques, such as linear regression, logistical regression, random forest, gradient boosted machine (GBM), decision tree, gradient boosting in a decision tree, deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for using companion pet DNA information to train a machine-learning model to predict a familial relationship of a companion pet, the method comprising:
receiving companion pet DNA information, the companion pet DNA information including at least one DNA sequence, at least one matching companion pet, and at least one familial label corresponding to the familial relationship between the companion pet and the at least one matching companion pet; and
upon the receiving, training the machine-learning model to predict at least one familial relationship from the at least one DNA sequence, the training further comprising:
for each of the at least one DNA sequence, extracting at least one local DNA fragment pattern from a database, the at least one local DNA fragment pattern corresponding to the at least one matching companion pet;
analyzing the at least one local DNA fragment pattern to determine at least one predicted familial relationship between the companion pet and the at least one matching companion pet; and
based on the analyzing, determining at least one predicted familial relationship and at least one corresponding confidence level.

2. The computer-implemented method of claim 1, wherein the at least one local DNA fragment pattern includes a plurality of IBD (identical-by-descent) fragments.

3. The computer-implemented method of claim 2, the analyzing further comprising:
determining a proportion of the at least one DNA sequence that contains the plurality of IBD fragments in only one of two sets of chromosomes.

4. The computer-implemented method of claim 2, the analyzing further comprising:
determining a proportion of the at least one DNA sequence that contains the plurality of IBD fragments in both of two sets of chromosomes.

5. The computer-implemented method of claim 2, the analyzing further comprising:
determining a proportion of the at least one DNA sequence that does not contain any of the plurality of IBD fragments.

6. The computer-implemented method of claim 2, the analyzing further comprising:
analyzing the plurality of IBD fragments to determine a total number of IBD fragments within the at least one local DNA fragment pattern.

7. The computer-implemented method of claim 2, the analyzing further comprising:
analyzing the plurality of IBD fragments to determine a total length of the plurality of IBD fragments within the at least one local DNA fragment pattern.

8. The computer-implemented method of claim 7, wherein the total length of the plurality of IBD fragments is measured in centimorgans.

9. The computer-implemented method of claim 1, the training further comprising:
determining that the at least one local DNA fragment pattern indicates an overlap of at least one trait; and
outputting a notification indicating that the at least one trait was shared with the at least one matching companion pet.

10. The computer-implemented method of claim 9, wherein the at least one trait is breed-specific.

11. The computer-implemented method of claim 1, wherein the at least one predicted familial relationship includes at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter.

12. The computer-implemented method of claim 1, wherein the companion pet DNA information further includes whether one or both sets of chromosomes match and a number of DNA fragments that match the at least one matching companion pet.

13. The computer-implemented method of claim 1, the determining further comprising:
outputting a list of matching DNA companion pet samples, wherein each of the matching DNA companion pet samples meet or surpass a matching threshold.

14. The computer-implemented method of claim 1, the training further comprising:
determining a homozygosity-by-descent level for the companion pet and for the at least one matching companion pet, the homozygosity-by-descent level indicating a level of inbreeding.

15. The computer-implemented method of claim 1, the analyzing further comprising:
analyzing the at least one local DNA fragment pattern to determine whether the at least one local DNA fragment pattern occurs on one set of chromosomes or both sets of chromosomes of the companion pet.

16. A computer system for using companion pet DNA information to train a machine-learning model to predict a familial relationship of a companion pet, the computer system comprising:
at least one memory storing instructions; and
at least one processor configured to execute the instructions to perform operations comprising:
receiving companion pet DNA information, the companion pet DNA information including at least one DNA sequence, at least one matching companion pet, and at least one familial label corresponding to a relationship between the companion pet and the at least one matching companion pet; and
upon the receiving, training the machine-learning model to predict at least one familial relationship from the at least one DNA sequence, the training further comprising:
for each of the at least one DNA sequence, extracting at least one local DNA fragment pattern from a database, the at least one local DNA fragment pattern corresponding to the at least one matching companion pet;
analyzing the at least one local DNA fragment pattern to determine at least one predicted familial relationship between the companion pet and the at least one matching companion pet; and
based on the analyzing, determining at least one predicted familial relationship and at least one corresponding confidence level.

17. The computer system of claim 16, the analyzing further comprising:
analyzing the at least one local DNA fragment pattern to determine whether the at least one local DNA fragment pattern occurs on one set of chromosomes or both sets of chromosomes of the companion pet.

18. The computer system of claim 16, wherein the at least one predicted familial relationship includes at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter.

19. A non-transitory computer-readable medium containing instructions that, when executed by a processor, cause the processor to perform operations for using companion pet DNA information to train a machine-learning model to predict a familial relationship of a companion pet, the operations comprising:
receiving companion pet DNA information, the companion pet DNA information including at least one DNA sequence, at least one matching companion pet, and at least one familial label corresponding to a relationship between the companion pet and the at least one matching companion pet; and
upon the receiving, training the machine-learning model to predict at least one familial relationship from the at least one DNA sequence, the training further comprising:
for each of the at least one DNA sequence, extracting at least one local DNA fragment pattern from a database, the at least one local DNA fragment pattern corresponding to the at least one matching companion pet;
analyzing the at least one local DNA fragment pattern to determine at least one predicted familial relationship between the companion pet and the at least one matching companion pet; and
based on the analyzing, determining at least one predicted familial relationship and at least one corresponding confidence level.

20. The non-transitory computer-readable medium of claim 19, wherein the at least one predicted familial relationship includes at least one of a mom, a dad, a sister, a brother, an uncle, an aunt, a niece, a nephew, a cousin, a grandfather, a grandmother, a grandson, a granddaughter, a great-grandfather, a great-grandmother, a great-grandson, a great-granddaughter, a half mom, a half dad, a half sister, a half brother, a half uncle, a half aunt, a half niece, a half nephew, a half cousin, a half grandfather, a half grandmother, a half grandson, a half granddaughter, a half great-grandfather, a half great-grandmother, a half great-grandson, or a half great-granddaughter.
